(19) United States
(12) Patent Application Publication
(10) Pub. No.: US 2026/0192026 A1
(43) Pub. Date: Jul. 9, 2026

(54) EXTRACORPOREAL BLOOD CIRCUIT PRIMING SOLUTION SAMPLING

(71) Applicant: Analog Devices International Unlimited Company, Limerick (IE)

(72) Inventors: Robert FINNERTY, Moate (IE); Claire E LEAHY, Listowel (IE)

(21) Appl. No.: 19/128,868

(22) PCT Filed: Nov. 10, 2023
(86) PCT No.: PCT/EP2023/081448

§ 371 Date: May 9, 2025

Related U.S. Application Data
(60) Provisional application No. 63/424,758, filed on Nov. 11, 2022.

(51) Int. Cl. A61M 1/16; A61M 1/36

(52) U.S. Cl. CPC A61M 1/1603; A61M 1/1698; A61M 1/3643; A61M 1/3666; A61M 2205/70

(57) ABSTRACT

The present disclosure provides a fluidic device for use in an extracorporeal bodily fluid system which comprises a sample flow path along which bodily fluid can flow and a flushing fluid unit which is configured to provide a biocompatible flushing fluid to the sample flow path in an upstream direction.

### FIELD OF THE DISCLOSURE

This disclosure relates to fluidic devices for use in an extracorporeal bodily fluid system, sensor systems for use in an extracorporeal bodily fluid systems and methods of operating a fluidic device.

### BACKGROUND

Extracorporeal bodily fluid systems, such as extracorporeal blood circuits, are used withdraw a bodily fluid from the patient's body before returning it back to the patient's body or to another patient. Such systems are used in the treatment systems which treat or modify the bodily fluid or may be used as a bypass while an organ is operated on. Examples of the former systems include dialysis (haemodialysis) and autotransfusion, and examples of the latter systems are a cardiopulmonary bypass (heart-lung machine) or extracorporeal membrane oxygenation (ECMO) device. Other extracorporeal bodily fluid systems include systems where a donor donates a bodily fluid (or a component of a bodily fluid) before it is transferred to a recipient.

Typically sensors are used in these circuits to monitor the bodily fluid. For example, these can be used to monitor levels of various analytes, such as ions, monitor temperature, pH, conductivity, etc. This can provide information on the patient's condition and the progress of any treatment. Some of these sensors are directly in-line in the circuit and continuously monitor the parameter. However, these have drawbacks, such as drift and a lack of sensitivity.

Other sensors sample a portion of the bodily fluid at particular intervals, for example by withdrawing a portion of the bodily fluid into a separate sampling fluid path. This can be advantageous as the sensing process can be carried out over a longer time period, increasing sensitivity and the sensors can be stored in calibration fluid between measurements, ensuring or improving accuracy.

However, these sensors can suffer from issues relating to the use of a bodily fluid, the lack of a continuous flow and the downtime between measurements. For example, bodily fluid such as blood can clot within the sensor fluidic pathways, such as the sampling fluid path, causing blockages and restrictions. Where valves are used, this can cause blockages of valves and failure of the sensor. Contamination can also occur.

It would be advantageous to provide a fluidic pathway which does not suffer from these issues.

### SUMMARY OF THE DISCLOSURE

The present disclosure provides a fluidic device for use in an extracorporeal bodily fluid system which comprises a sample flow path along which bodily fluid can flow and a flushing fluid unit which is configured to provide a biocompatible flushing fluid to the sample flow path in an upstream direction.

In one embodiment, a fluidic device for use in an extracorporeal bodily fluid system comprising a circulatory fluid flow path comprises: a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly; a sampling valve provided along the sample flow path, the sampling valve configured to selectively permit fluid to flow along the sample flow path between the inlet and the sensor assembly interface; and a flushing unit for flushing bodily fluid from at least a part of the sample flow path. The flushing unit comprises a flushing fluid reservoir for receiving a biocompatible flushing fluid, the flushing fluid reservoir fluidically connected to the sample flow path at a first junction downstream of the inlet; and a flushing fluid delivery unit configured to provide the biocompatible flushing fluid to the sample flow path from the flushing fluid reservoir. The fluidic device is configured such that, in use, the flushing fluid delivery unit causes biocompatible flushing fluid to flow upstream from the flushing fluid reservoir to the inlet of the sample flow path.

In one embodiment, a sensor system for use in an extracorporeal bodily fluid sensing system comprises a fluidic device according to any of the embodiments set out herein; and a control unit configured to operate the fluidic device, wherein the control unit is configured to operate the fluidic device so as to cause biocompatible flushing fluid to flow upstream from the flushing fluid reservoir to the inlet of the sample flow path. The control unit accordingly can be configured to control the flushing device and the sampling valve, and may further control operation of any further devices within the flow path or in connection with the flow path, such as valves or pumping mechanisms.

In one embodiment, a method of operating a fluidic device comprises:
providing a fluidic device comprising: a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly; a sampling valve provided along the sample flow path, the sampling valve configured to selectively permit fluid to flow along the sample flow path between the inlet and the sensor assembly interface; and a flushing unit for flushing bodily fluid from at least a part of the sample flow path, the flushing unit comprising: a flushing fluid reservoir for receiving a biocompatible flushing fluid, the flushing fluid reservoir fluidically connected to the sample flow path at a first junction downstream of the inlet; and a flushing fluid delivery unit configured to provide the biocompatible flushing fluid to the sample flow path from the flushing fluid reservoir,
flowing bodily fluid from the inlet to the sensor assembly interface of the sample flow path; and
flushing the sample flow path to remove bodily fluid by flowing biocompatible flushing fluid to flow upstream from the flushing fluid reservoir to the inlet of the sample flow path.

### BRIEF DESCRIPTION OF THE DRAWINGS

The present invention will now be described in more detail with reference to the accompanying drawings, which are not intended to be limiting:

FIG. 1 provides a schematic plan view of a fluidic device according to an embodiment;

FIG. 2 provides a schematic plan view of a sensor system according to an embodiment;

FIGS. 3A to 3D provides a schematic plan view of the fluidic device of FIG. 1 in use in an embodiment;

FIGS. 4A and 4B provides a schematic plan view of the fluidic device of FIG. 1 in use in an embodiment;

FIG. 5 provides a schematic depiction of an extracorporeal bodily fluid device in use;

FIG. 6 schematic plan view of a fluidic device according to an embodiment; and

FIG. 7 depicts a method according to an embodiment.

### DETAILED DESCRIPTION

Extracorporeal bodily fluid circuits, such as extracorporeal blood circuits, are used withdraw a bodily fluid from the patient's body before returning it back to the body or to the body of another patient. These circuits incorporate or use sensors to monitor the bodily fluid. One sensor type is used in a system which samples a portion of the bodily fluid at intervals by withdrawing a portion of the bodily fluid into a separate sample flow path. These types of sensors can suffer from issues relating to the use of a bodily fluid, the lack of a continuous flow and the downtime between measurements. For example, bodily fluid such as blood can clot within the sensor fluidic pathways, such as in dead space in the sampling fluid path or valves, causing blockages and restrictions. Where valves are used, this can cause blockages of valves and failure of the sensor. Contamination can also occur.

It would be advantageous to provide a fluidic pathway which does not suffer from these issues.

In one embodiment, a fluidic device for use in an extracorporeal bodily fluid system comprising a circulatory fluid flow path comprises: a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly; a sampling valve provided along the sample flow path, the sampling valve configured to selectively permit fluid to flow along the sample flow path between the inlet and the sensor assembly interface; and a flushing unit for flushing bodily fluid from at least a part of the sample flow path. The flushing unit comprises a flushing fluid reservoir for receiving a biocompatible flushing fluid, the flushing fluid reservoir fluidically connected to the sample flow path at a first junction downstream of the inlet; and a flushing fluid delivery unit configured to provide the biocompatible flushing fluid to the sample flow path from the flushing fluid reservoir. The fluidic device is configured such that, in use, the flushing fluid delivery unit causes biocompatible flushing fluid to flow upstream from the flushing fluid reservoir to the inlet of the sample flow path.

Embodiments provide a fluidic device which avoids the aforementioned problems with bodily fluid build-up and contamination in parts of the sample flow path (e.g. in the flow path or in valves), and associated flow paths, and therefore improves the reliability of the fluidic device and associated sensor.

In particular, the device is configured (or operable) such that bodily fluid in at least the upstream part of the sample flow path is expelled from the sample flow path to the circulatory fluid flow path. This enables the regions of the fluidic device and sensor assembly which are in open and direct contact with the bodily fluid in the circulatory fluid flow path (e.g. the upstream-side of the sampling valve and any flow paths upstream of the sampling valve) which will be returned to the patient to be cleaned and residue or contaminants, such as clotted blood, to be removed. This has significant advantages compared to devices which provide an associated sensor assembly with a calibration fluid or flush fluid which are only used to clear a downstream portion and would otherwise risk device failure (e.g. valve failure) due to clotting or clogging or may otherwise risk contamination of further samples. In many cases, extracorporeal bodily fluid systems will be connected to a patient for many hours, even days, and continuous automatic sampling and sensing is required without failure.

It will be appreciated that strict regulations on contamination and patient health mean that only regulator-approved fluids can be introduced into a patient. Embodiments provide a fluidic device which has a fluid reservoir which can store biocompatible fluid (e.g. a sterile reservoir) and is configured so that this is provided in the upstream direction (e.g. through the use of valves), such that a biocompatible fluid can clear the entire upstream flow path without fear of contaminating a patient's bodily fluid with an unsuitable fluid. That is, the biocompatible fluid here may be clear out the upstream part of the sample flow path and any upstream-facing components or surfaces, before it is safely returned to the circuit and subsequently returned to the patient. The latter also reduces the capacity required to store biowaste, which would otherwise be diverted to waste.

The fluidic device is for an extracorporeal bodily fluid system comprising a circulatory fluid flow path. By circulatory fluid flow path in extracorporeal bodily fluid sensing system, it is meant a fluid flow path which extracts a bodily fluid from the patient and then returns the fluid to the patient (e.g. in a circulatory manner). An extracorporeal bodily fluid system may also use a fluid flow path which is used to extract a bodily fluid from the patient and returns it to another. The bodily fluid may be treated or modified during circulation. The circulatory fluid flow path may be comprised of several flow paths, in parallel and/or series, and may include passing the bodily fluid through treatment devices or systems. Examples of systems incorporating such circulatory fluid flow paths include dialysis or extracorporeal membrane oxygenation (ECMO) systems. In embodiments, there is provided an extracorporeal bodily fluid sensing system comprising a fluidic device as disclosed herein and a circulatory fluid flow path in fluid communication with the fluidic device.

In some embodiments, the bodily fluid is blood. Examples of systems extracting and returning blood include dialysis (haemodialysis) and autotransfusion, cardiopulmonary bypass (heart-lung machine) or extracorporeal membrane oxygenation (ECMO) device. Blood being returned to a patient is subject to stringent requirements and should not be compromised, for example by contamination with a cleaning fluid.

The inlet is connectable to such a circulatory fluid flow path so as to receive bodily fluid therefrom. Sampling from the circulatory fluid flow path may be at discrete time intervals and may be automated. The sensor assembly interface may be an outlet, for example an outlet for or in fluid connection with a sensor assembly, or may be a portion of the sample flow path. The latter may be a part of the sample flow path with openings or apertures in fluid communication with a sensor assembly or it may be that a sensor assembly is received within this part of the sample flow path (forming the sensor assembly interface).

The fluidic device uses a flushing fluid which is a biocompatible fluid; that is, a fluid (e.g. a liquid) which can safely be provided to a patient's body (in a mixture with the bodily fluid) and is not harmful to the patient. In one embodiment, the biocompatible fluid is a saline solution or an infusion liquid.

In one embodiment, the first junction is downstream of the sampling valve in the sample flow path. By downstream, it is meant towards the sensor assembly interface, such that the first junction is fluidly connected to the sample flow path at a point between the sampling valve and the sensor assembly interface. This means that, in use, the flushing fluid delivery unit causes biocompatible flushing fluid to flow from the flushing fluid reservoir in an upstream direction along the sample fluid flow path to and through the inlet of the sample flow path through the sampling valve. Valves have moveable parts through which the bodily fluid will flow and accordingly are at risk of contamination and clogging. For example, blood left in dead space in the valves is prone to clotting and can cause the valve to seize. Passing the flushing fluid through the valve will clear the valve and reduce the risk of failure. Moreover, flushing in this manner is preferential over flushing systems using downstream flushing systems only, since it allows the upstream surfaces of the valve to be cleaned and the valve to be cleaned in an open position, ensuring all of the surfaces which will contact the bodily fluid can be cleaned. Although the downstream part of the valve could be cleaned by a downstream fluid, the upstream parts could not and the valve would need to remain closed to prevent return of this non-biocompatible calibration fluid. Moreover, these embodiments are advantageous as only the patient-facing surfaces need to be cleared with biocompatible fluid, and less costly or more optimal fluids for flushing and/or calibration can be used in the remainder of the fluidic device and sensor assembly (e.g. with the sampling valve closed).

In one embodiment, the fluidic device further comprises a sensor assembly control valve provided between the first junction and the sensor assembly interface, the sensor assembly control valve configured to selectively permit fluid to flow along the sample flow path between the first junction and the sensor assembly interface. In other words, the sensor assembly control valve is provided downstream of the first junction (but upstream of the sensor assembly interface).

In embodiments, the fluidic device is configured such that, in use, the sample assembly control valve is closed to prevent flushing fluid flowing to the sensor assembly interface. This accordingly prevents flow to an associated sensor assembly. This can help to speed up the flushing process and helps to ensure separation of any calibration fluid on the sensor assembly and the bodily fluids which will be returned to the circulatory fluid flow path.

These embodiments also have the advantage of enabling the biocompatible reservoir and sample flow path upstream of the sensor assembly control valve to be separated from the downstream components. A non-biocompatible fluid (e.g. a calibration fluid or flush fluid) can then be used to clear the fluidic parts downstream of the sensor assembly control valve, reducing the volume requirement for the biocompatible fluid (and thus cost and complexity of storage).

In one embodiment, the device further comprises a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path. The fluidic device can be configured such that, in use, the calibration fluid delivery unit causes calibration fluid to flow from the calibration fluid reservoir downstream to the sensor assembly interface of the sample flow path. The device may further comprise a calibration fluid reservoir valve at the second junction and configured to control flow of calibration fluid from the calibration fluid reservoir to the sample flow path.

In one embodiment, the first junction is upstream of the second junction. In other words, the flushing fluid reservoir of the flushing unit is fluidically connected to the sample flow path at a position upstream of the position at which the calibration fluid reservoir is fluidically connected to the sample flow path. This reduces the risk that calibration fluid, such as any caught in dead space around the second junction or along the sample flow path, will be flushed into the circulatory fluid flow path and thus contaminate the bodily fluid.

In one embodiment, a valve is located in the sample flow path between the first and second junctions to selectively permit fluid flow along the sample flow path. In some embodiments, this may be the sampling valve. In other embodiments, this may be an additional valve.

In one embodiment, the flushing unit is operable to draw fluid from the sample flow path into the flushing fluid reservoir. Prior to use with a patient, extracorporeal bodily fluid sensing systems are typically filled with a biocompatible fluid, such as a saline solution. This biocompatible fluid is also typically provided to the fluidic device of associated sensing systems. By providing a flushing unit which is operable (configured such that it can) draw fluid from the sample flow path, it is possible to draw this biocompatible fluid into the reservoir and subsequently use this stored fluid for the flushing process. This avoids having to store a biocompatible fluid within the fluidic device itself prior to use, which would increase the complexity and cost of manufacture significantly and may require regulatory approval for the fluidic device. In other embodiments, this may be drawn up from a separate source prior to use, such as a saline bag. These embodiments still benefit from not having to provide the fluidic device with biocompatible fluid during manufacture, since the biocompatible fluid can be taken at the point of use. In alternative or additional embodiments, the flushing fluid reservoir may be provided with a flushing fluid during manufacture, such as the flushing fluid reservoir may comprise the biocompatible flushing fluid. In some specific embodiments, the flushing unit may comprise an actuator configured to draw fluid from the sample flow path, such as a pump or syringe.

In one embodiment, the fluidic device further comprises a sensor assembly, wherein the sensor assembly interface of the sample flow path is in fluid communication with the sensor assembly. The fluidic device is therefore configured such that fluid, such as bodily fluid or calibration fluid, provided to the sample flow path can be provided to the sensor assembly through the sensor assembly interface. The sensor assembly comprises at least one sensor (or transducer) configured to measure a property of fluid provided thereto.

In one embodiment, the fluidic device is further configured such that, in use, the flushing fluid delivery unit causes biocompatible flushing fluid to flow downstream from the flushing fluid reservoir through sample flow path. This may be to the sensor assembly interface or to another outlet, such as a waste reservoir. Washing in both directions ensures that valves are cleaned in all positions thereby further reducing the risk of contamination.

In one embodiment, a sensor system for use in an extracorporeal bodily fluid sensing system comprises a fluidic device according to any of the embodiments set out herein; and a control unit configured to operate the fluidic device, wherein the control unit is configured to operate the fluidic device so as to cause biocompatible flushing fluid to flow upstream from the flushing fluid reservoir to the inlet of the sample flow path. The control unit accordingly can be configured to control the flushing device and the sampling valve, and may further control operation of any further devices within the flow path or in connection with the flow path, such as valves or pumping mechanisms.

The control unit accordingly configures the fluidic device in such a way to cause biocompatible flushing fluid to flow upstream from the flushing fluid reservoir to the inlet of the sample flow path, for example operating any valves between the inlet and the flushing fluid reservoir. This may also comprise closing other valves so that the flushing fluid does not travel downstream and, for example, mix with any calibration fluid in the sample flow path and sensor assembly.

The control unit may be implemented in any suitable manner, for example with software and/or hardware, to perform the various functions required. This may, for example, employ one or more microprocessors programmed using software (for example, microcode) to perform the required functions. Examples of processor components that may be employed in various embodiments include, but are not limited to, conventional microprocessors, application specific integrated circuits (ASICs), and field-programmable gate arrays (FPGAs). In various implementations, the control unit may be associated with one or more non-transitory storage media such as volatile and non-volatile computer memory such as RAM, PROM, EPROM, and EEPROM. The non-transitory storage media may be encoded with one or more programs that, when executed on one or more processors and/or controllers, perform the required functions. Various storage media may be fixed within a processor or controller or may be transportable, such that the one or more programs stored thereon can be loaded into the control unit.

In one embodiment, a computer program comprising computer program code may be configured, when said computer program is run on one or more physical computing devices, to cause said one or more physical computing devices to implement the methods or functions of the control unit disclosed herein. In one embodiment, one or more non-transitory computer readable media having a computer program stored thereon, the computer program comprising computer program code which is configured, when said computer program is run on one or more physical computing devices, may cause aid one or more physical computing devices to implement the methods or functions of the control unit disclosed herein.

In one embodiment, the fluidic device further comprises a sensor assembly control valve provided between the first junction and the sensor assembly interface, the sensor assembly control valve configured to selectively permit fluid to flow along the sample flow path between the first junction and the sensor assembly interface. In such embodiments, the control unit can be configured to close the sensor assembly control valve when operating the fluidic device to provide biocompatible flushing fluid to flow upstream from the flushing fluid reservoir to the inlet of the sample flow path.

In an embodiment, the control unit is configured to operate the sampling valve to provide bodily fluid from the inlet to the sensor assembly interface; and subsequently, the control unit is configured to operate the fluidic device so as to cause biocompatible flushing fluid to flow upstream from the flushing fluid reservoir to the inlet of the sample flow path. This enables the clearing of any bodily fluid from the sample flow path. This can be after the sampling valve has been closed.

In one embodiment, the flushing unit is operable to draw fluid from the sample flow path into the flushing fluid reservoir; and the control unit is configured to operate the fluidic device so as to draw fluid from the sample flow path into the flushing fluid reservoir.

In one embodiment, the system further comprises a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path; and the control unit is configured to control delivery of the calibration fluid to the sensor assembly interface. This can be a sensor assembly, when present. The control unit may be configured to prevent flow of the calibration fluid to the inlet and therefore may be configured to control operation of any upstream valves to close at least one of these valves (such as the sampling valve).

In one embodiment, the control unit is configured to operate the fluidic device so as to cause biocompatible flushing fluid to flow downstream from the flushing fluid reservoir through sample flow path. This may be to the sensor assembly interface or to another outlet, such as a waste reservoir. Washing in both directions ensures that valves are cleaned in all positions thereby further reducing the risk of contamination.

In one embodiment, a method of operating a fluidic device comprises:
providing a fluidic device comprising: a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly; a sampling valve provided along the sample flow path, the sampling valve configured to selectively permit fluid to flow along the sample flow path between the inlet and the sensor assembly interface; and a flushing unit for flushing bodily fluid from at least a part of the sample flow path, the flushing unit comprising: a flushing fluid reservoir for receiving a biocompatible flushing fluid, the flushing fluid reservoir fluidically connected to the sample flow path at a first junction downstream of the inlet; and a flushing fluid delivery unit configured to provide the biocompatible flushing fluid to the sample flow path from the flushing fluid reservoir,
   flowing bodily fluid from the inlet to the sensor assembly interface of the sample flow path; and
   flushing the sample flow path to remove bodily fluid by flowing biocompatible flushing fluid to flow upstream from the flushing fluid reservoir to the inlet of the sample flow path.

In one embodiment, the fluidic device can be a fluidic device as set out in accordance with any of the embodiments disclosed herein.

In one embodiment, prior to the step of flowing bodily fluid from the inlet to the sensor assembly interface of the sample flow path, the method further comprises providing a biocompatible fluid to the sample flow path; and drawing the biocompatible fluid from the sample flow path into the flushing fluid reservoir, wherein the biocompatible fluid is used in the subsequent step of flushing the sample flow path to remove bodily fluid. As set out above, prior to use with a patient, extracorporeal bodily fluid sensing systems are typically filled with a biocompatible fluid, such as a saline solution. This biocompatible fluid is also typically provided to the fluidic device of associated sensing systems. By providing a flushing unit which is operable (configured such that it can) draw fluid from the sample flow path, it is possible to draw this biocompatible fluid into the reservoir and subsequently use this stored fluid for the flushing process. This avoids having to store a biocompatible fluid within the fluidic device itself prior to use, which would increase the complexity and cost of manufacture significantly and may require regulatory approval for the fluidic device.

In one embodiment, the method further comprises providing a sensor assembly fluidly connected to the sensor assembly interface of the sample flow path; and the step of flowing bodily fluid from the inlet to the sensor assembly interface of the sample flow path further comprises flowing the bodily fluid over the sensor assembly so that a property of the bodily fluid can be detected. The method may further comprise detecting a property of the bodily fluid, such as the concentration of one or more analytes (e.g. ions).

In one embodiment, the fluidic device further comprises a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path; and the method further comprises providing calibration fluid to the sensor assembly interface. This can be prior to measurement and/or after measurement.

In one embodiment, flushing the sample flow path to remove bodily fluid further comprises flowing biocompatible flushing fluid to flow downstream from the flushing fluid reservoir through sample flow path. This may be to the sensor assembly interface or to another outlet, such as a waste reservoir.

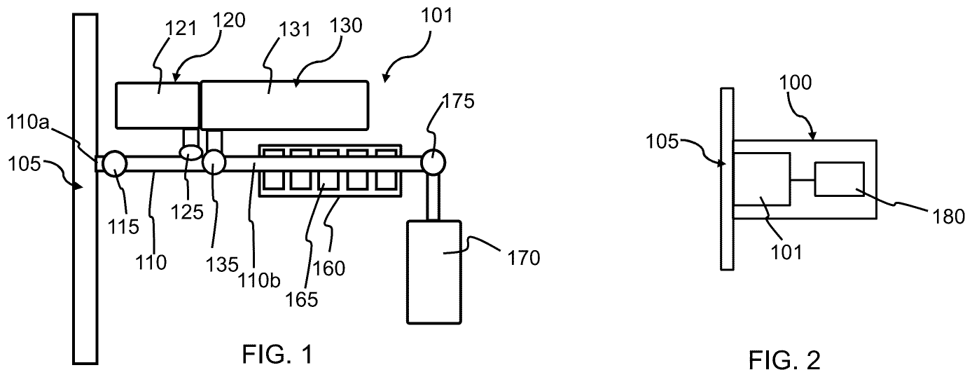

FIG. 1 provides a schematic plan view of a fluidic device 101 for use in an extracorporeal bodily fluid system, the extracorporeal bodily fluid system comprising a circulatory fluid flow path 105 in fluid communication with the fluidic device 101. Specifically, the extracorporeal bodily fluid system is one for treatment of a patient in which a bodily fluid BF, such as blood, is received from a patient into the circulatory fluid flow path 105, is treated or modified by the extracorporeal bodily fluid system and is subsequently returned to the patient via the circulatory fluid flow path 105. The fluidic device 101 is provided to sample bodily fluid BF from the circulatory fluid flow path 105 and is used to determine at least one property of the bodily fluid BF.

The fluidic device 101 comprises a sample flow path 110 comprising an inlet 110a at one end of the sample flow path 110, which inlet 110a is in fluid connection with the circulatory fluid flow path 105, and a sensor assembly interface 110b downstream of the inlet 110a. In this way, the inlet 110a can receive bodily fluid BF from the circulatory fluid flow path 105, which bodily fluid BF can then flow along the sample flow path 110 to the sensor assembly interface 110b. In this embodiment, the fluidic device 101 further comprises a sensor assembly 160 comprising a plurality of sensors 165 for detecting and measuring a property of various analytes (e.g. concentration). Examples include sensors 165 for detecting the concentration of particular ions, conductivity, pH and temperature. The sample flow path 110 passes over the sensor assembly 160 and has a series of apertures (not shown) corresponding to each of the plurality of sensors 165 so that fluid (such as bodily fluid BF) can come into contact with the plurality of sensors 165, thereby forming sensor assembly interface 110b. In this embodiment, the sample flow path 110 continues to an outlet in communication with waste reservoir 170, where it is discharged.

The fluidic device 101 further comprises a sampling valve 115 provided along the sample flow path 110, and in particular arranged upstream of the sensor assembly interface 110b and provided adjacent the inlet 110a. The sampling valve 115 is configured to selectively permit fluid, such as bodily fluid BF from the circulatory fluid flow path 105, to flow along the sample flow path 110 between the inlet 110a and the sensor assembly interface 110b.

The fluidic device 101 also comprises a flushing fluid delivery unit 120 for flushing bodily fluid BF from at least a part of the sample flow path 110. The flushing fluid delivery unit 120 comprises a flushing fluid reservoir 121 which receives a biocompatible flushing fluid FF and a flushing fluid delivery unit configured to provide the biocompatible flushing fluid FF to the sample flow path 110. In this embodiment, the flushing fluid delivery unit is an actuator (such as a pump) integrated into the flushing fluid reservoir 121 (and therefore not depicted separately). The flushing fluid delivery unit 120 is joined to the sample flow path 110 at a first junction between the inlet 110a and the sensor assembly interface 110b and comprises a flushing unit valve 125 provided between the flushing fluid reservoir 121 and the sample flow path 110 which selectively permits fluid flow between the flushing fluid reservoir 121 and the sample flow path 110. The flushing fluid delivery unit 120, and specifically the flushing fluid delivery unit, is configured to provide the biocompatible flushing fluid FF to the sample flow path 110 from the flushing fluid reservoir 121.

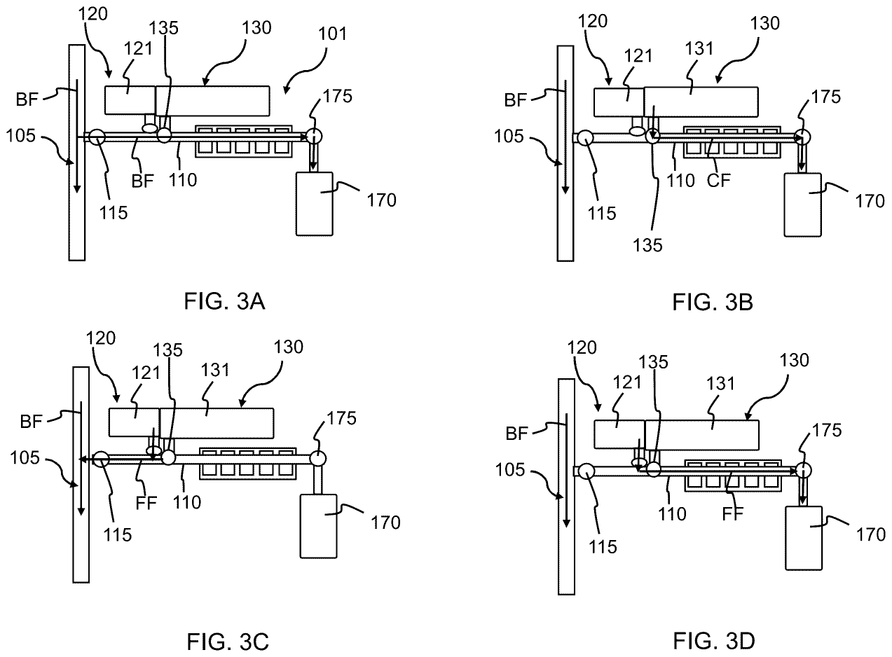

As can be seen from FIG. 1 and the above description of the sampling valve 115 and the relative location of the flushing fluid delivery unit 120 to the sample flow path 110, the fluidic device 101 is configured such that, in use, the flushing fluid delivery unit 120 is operable to cause biocompatible flushing fluid FF to flow upstream from the flushing fluid reservoir 121 to the inlet 110a of the sample flow path 110. This is described with reference to FIGS. 3A to 3D, below.

The fluidic device 101 in this embodiment further comprises a calibration fluid unit 130 comprising a calibration fluid reservoir 131 for receiving a calibration fluid CF. The calibration fluid reservoir 131 is fluidically connected to the sample flow path 110 at a second junction, specifically between the sampling valve 115 and the sensor assembly interface 110b, in this embodiment, downstream of the first junction. In this embodiment, a sensor assembly control valve 135 is provided at the second junction and acts as a three-way valve controlling flow of calibration fluid CF from the calibration fluid reservoir 131 to the sensor assembly interface 110b, and thus the sensor assembly 160, flow of fluid along the sample flow path 110 between the inlet 110a and the sensor assembly interface 110b. The calibration fluid unit 130 also comprises a calibration fluid delivery unit configured to provide the calibration fluid CF to the sample flow path 110. In this embodiment, the calibration fluid delivery unit is an actuator (such as a pump) integrated into the calibration fluid reservoir 131 (and therefore not depicted separately).

The calibration fluid CF is a fluid provided for ensuring that the sensors 165 of the sensor assembly 160 are accurate and do not drift during use. When not carrying out measurements, the sensors 165 will usually be immersed in the calibration fluid CF. The calibration fluid CF will also therefore have a cleaning function for the sensor assembly 160; however, it is not usually a fluid that is biocompatible and so it cannot re-enter the circulatory fluid flow path 105. For example, the calibration fluid CF is usually a sensor assembly-specific fluid designed to have particular concentrations of analytes (e.g. ions) and will not typically be approved by a regulator for use in a patient's body. Such an approval process is onerous, country or region-specific and would need to be specific for each calibration fluid, which may differ depending on the specific sensor array. Accordingly, avoiding having to obtain approval for this is a significant advantage. The fluidic device 101 avoids this by using the flushing fluid delivery unit 120, as set out below, and by having the sensor assembly control valve 135 which can prevent fluid flow upstream from the calibration fluid reservoir 131.

Downstream of the sensor assembly interface 110b and sensor assembly 160 is a waste reservoir 170 for receiving any waste fluid. Access to this is controlled by a waste valve 175, which selectively allows for fluid flow out of the sample flow path 110 and into the waste reservoir 170.

FIG. 2 provides a schematic plan view of a sensor system 100 for use in an extracorporeal bodily fluid system. The sensor system 100 comprises the fluidic device 101 of FIG. 1 which is in communication with a control unit 180 configured to operate the fluidic device 101. Specifically, the control unit 180 is configured to operate the fluidic device 101 so as to, amongst other possible operations, cause biocompatible flushing fluid FF to flow upstream from the flushing fluid reservoir 121 to the inlet 110a of the sample flow path 110. This is through operation of the various valves and delivery units in the fluidic device 101 to provide this, as will be described in more detail below.

FIGS. 3A to 3D schematically depict the operation of the fluidic device 101 of FIG. 1 in one embodiment, which may be under the control of the control unit 180 as part of the sensor system 100 depicted in FIG. 2. That is, each of the operations may be controlled by the control unit 180.

FIG. 3A depicts the fluidic device 101 during a sensing operation. In this configuration, the sampling valve 115 and the sensor assembly control valve 135 are opened so that the sample flow path 110 is open from the inlet 110a to the sensor assembly 160 and bodily fluid BF can flow from the circulatory fluid flow path 105 to sensor assembly 160. This is typically under the pressure caused by flow of the bodily fluid BF within the circulatory fluid flow path 105. With bodily fluid BF in contact with the sensors 165 of the sensor assembly 160, the sensor system 100 can measure properties of the bodily fluid BF. Although not depicted, the sampling valve 115 and/or the sensor assembly control valve 135 may be closed during measurement and the bodily fluid BF may be left to reside on the sensors 165. Alternatively, there may be a continuous flow of bodily fluid BF during this process.

FIG. 3B depicts the fluidic device 101 after a measurement operation and undergoing a cleaning operation. In a first step, calibration fluid CF is delivered from the calibration fluid reservoir 131 to the sample flow path 110 and downstream to the sensor assembly 160 and ultimately the waste reservoir 170 (via waste valve 175, which may be open for at least a part of this step). In this embodiment, the sensor assembly control valve 135 prevents flow upstream of the sensor assembly control valve 135 but allows calibration fluid CF from the calibration fluid reservoir 131 to flow through the sensor assembly control valve 135 and downstream. This process clears these portions of the fluidic device 101 of bodily fluid BF and is used to recalibrate the sensors between measurements.

FIG. 3C depicts a further step in the cleaning operation, which may be carried out simultaneously to that depicted in FIG. 3B or separately to this step (e.g. before or after). In this step, with the sensor assembly control valve 135 closed and the sampling valve 115 open (for at least part of the process), the flushing fluid delivery unit causes biocompatible flushing fluid FF to flow from the flushing fluid reservoir 121 upstream to the inlet 110a of the sample flow path 110 and out through the inlet 110a into the circulatory fluid flow path 105 of the extracorporeal system. This cleans all of the upstream parts of the sample flow path 110 and associated valves, thereby reducing build-up of components of bodily fluids BF within these channels and on any component within this part of the fluid flow paths. For example, this allows the sampling valve 115 to be cleaned, including the circulatory fluid flow path 105 side of the sampling valve 115 which would otherwise be in contact with bodily fluid BF only, as well as any dead space within the upstream portion of the sample flow path 110. As set out above, this is particularly advantageous as it reduces fluidic device 101 failure or inaccuracies (e.g. due to contamination) by reaching areas that cannot be otherwise cleaned, for example by calibration fluid CF. As the flushing fluid FF is a biocompatible fluid, it can be returned to the circulatory fluid flow path 105, and therefore the patient, without risk of harming the patient or significantly impacting the properties of the bodily fluid BF in a detrimental manner.

FIG. 3D depicts a further washing step, which may be carried out in addition to or instead of the washing step depicted to in FIG. 3B. In this step, the fluidic device 101 is configured to flow flushing fluid FF from the flushing fluid reservoir 121 downstream through the sensor assembly interface 110b and the sensor assembly 160 to the waste reservoir 170. This allows for the flushing fluid FF to clean the sensor assembly control valve 135 in the open position, such that it can be fully cleaned as well as any associated dead space. For example, this may clean any dead space between the first junction and the most upstream part cleaned by the calibration fluid CF (i.e. anything downstream of the first junction but which may not be reached by the calibration fluid (i.e. upstream of the sensor assembly control valve 135) but not within the main flow of flushing fluid FF between the flushing fluid reservoir 121 and the inlet 110a). The flushing unit valve 125 and the sensor assembly control valve 135 are accordingly open for at least a part of this operation. The waste valve 175 may also be open for at least a part of this. In this step, the sampling valve 115 may be open or closed. This may be closed to encourage flushing fluid FF to flow to the waste reservoir 170, for example.

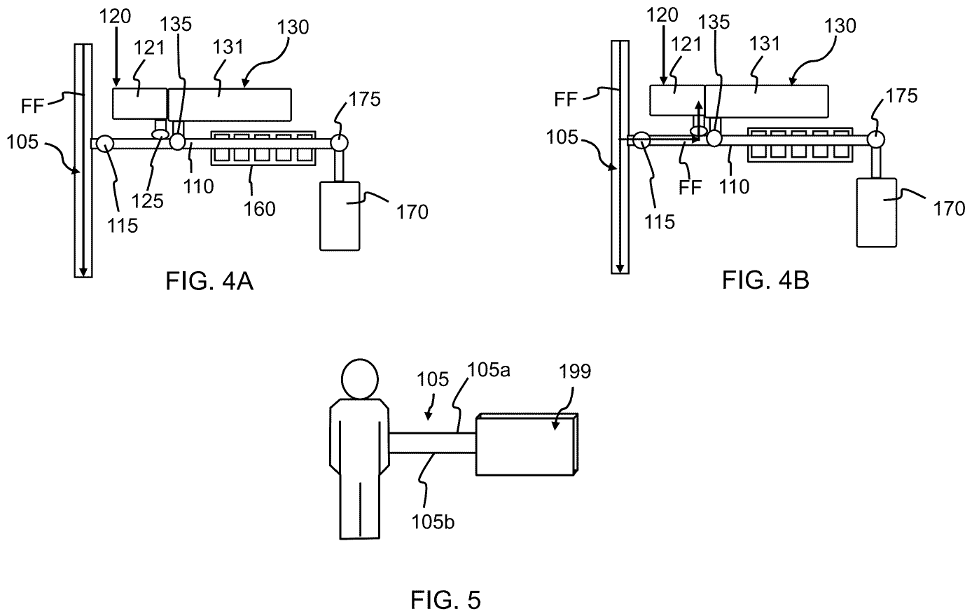

FIGS. 4A and 4B schematically depict a further operation of the fluidic device 101 of FIG. 1 in one embodiment, which may be under the control of the control unit 180 as part of the sensor system 100 depicted in FIG. 2. That is, each of the operations may be controlled by the control unit 180. The operations schematically depicted in FIGS. 4A and 4B may be carried out in addition to those steps schematically depicted in FIGS. 3A to 3D and described above, for example prior to the steps schematically depicted in FIGS. 3A to 3D and described above.

FIG. 4A depicts the fluidic device 101 and associated circulatory fluid flow path 105 of an extracorporeal bodily fluid system in an initial state whereby no bodily fluid BF has been introduced into the circulatory fluid flow path 105. Instead, the extracorporeal bodily fluid system, including the circulatory fluid flow path 105, has been primed with a biocompatible fluid, such as a saline solution, which can be used as a biocompatible flushing fluid FF. This is typically the case prior to use of an extracorporeal bodily fluid system. In this embodiment and in the configuration depicted in FIG. 4A, the flushing fluid reservoir 121 is empty (i.e. has no biocompatible flushing fluid FF therein).

To provide the fluidic device 101 with biocompatible flushing fluid FF, the fluidic device 101 is operated so as to draw the biocompatible flushing fluid FF into the flushing fluid reservoir 121. Specifically, in this embodiment, prior to the step of flowing bodily fluid BF through the circulatory fluid flow path 105 and the sample flow path 110, the sampling valve 115 is opened and the flushing fluid delivery unit 120 is operated so as to open the flushing unit valve 125 and the actuator located in the flushing fluid reservoir 121 draws the biocompatible fluid from the sample flow path into the flushing fluid reservoir. The biocompatible fluid thus becomes the biocompatible flushing fluid FF. The fluidic device 101 can now be used in the manner set out above, for example, as depicted in FIGS. 3A to 3B, such that the stored biocompatible flushing fluid FF can be used in subsequent steps of flushing the sample flow path 110 to remove bodily fluid BF.

Such embodiments are advantageous as these avoid the need to store a biocompatible fluid within the fluidic device itself prior to use, which can increase the complexity and cost of manufacture significantly and may require regulatory approval for the fluidic device.

As set out above, operation of the fluidic device 101 in the manners schematically depicted in FIGS. 3A to 3D, 4A and 4B may be under the control of the control unit 180. Specifically, the control unit 180 may be configured to control the opening and closing of the sampling valve 115, the sensor assembly control valve 135 and/or the waste valve 175, as well as operation of the flushing fluid delivery unit and the calibration fluid delivery unit.

FIG. 5 depicts an extracorporeal bodily fluid system 199 in use, with a patient connected to the circulatory fluid flow path 105. Specifically, the circulatory fluid flow path includes an inlet portion or line 105a and an outlet portion or line 105b which extract and return the bodily fluid BF, respectively. The fluidic device 101 may be housed within the extracorporeal bodily fluid system 199 or may be a separate component attached thereto. The fluidic device 101 may be attached to any part of the circulatory fluid flow path 105, for example, including the inlet portion or line 105a or an outlet portion or line 105b.

Although in the embodiments, the fluidic device 101 and control unit 180 are in physical connection, it will be appreciated that any implementation of a control unit 180 is possible and this may be remote from the fluidic device 101.

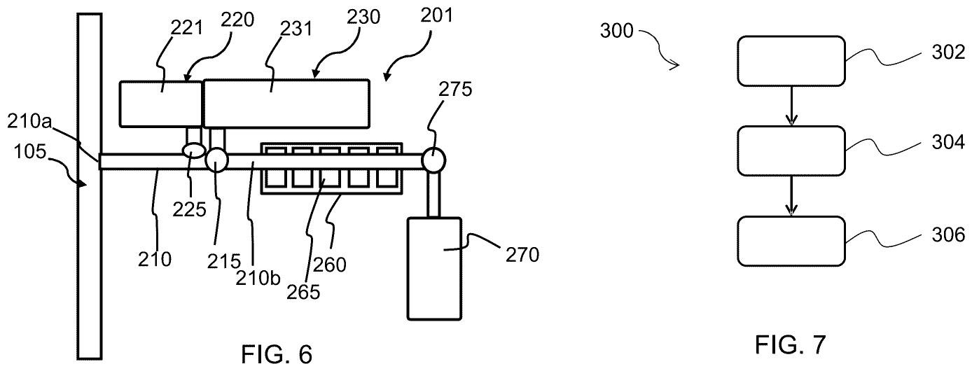

FIG. 6 provides a schematic plan view of another embodiment of a fluidic device 201 for use in an extracorporeal bodily fluid system. It differs from the fluidic device 101 of FIG. 1 in the order of the components in the sample flow path. As with the embodiment of FIG. 1, the fluidic device 201 connects to a circulatory fluid flow path 105.

The fluidic device 201 comprises a sample flow path 210 comprising an inlet 210a at one end of the sample flow path 210, which inlet 210a is in fluid connection with the circulatory fluid flow path 105, and a sensor assembly interface 210b downstream of the inlet 210a. The fluidic device 201 further comprises a sensor assembly 260 comprising a plurality of sensors 265 for detecting and measuring a property of various analytes (e.g. concentration). The sample flow path 210 passes over the sensor assembly 260 and has a series of apertures (not shown) corresponding to each of the plurality of sensors 265 so that fluid (such as bodily fluid BF) can come into contact with the plurality of sensors 265, thereby forming sensor assembly interface 210b. In this embodiment, the sample flow path 210 continues to an outlet in communication with waste reservoir 270, where fluid can be discharged.

The fluidic device 201 further comprises a sampling valve 215 provided along the sample flow path 210 and arranged upstream of the sensor assembly interface 210b. In this embodiment, the sampling valve is provided adjacent the sensor assembly interface 210b. The sampling valve 215 is configured to selectively permit fluid, such as bodily fluid BF from the circulatory fluid flow path 105, to flow along the sample flow path 210 between the inlet 210a and the sensor assembly interface 210b.

The fluidic device 201 also comprises a flushing fluid delivery unit 220 for flushing bodily fluid BF from at least a part of the sample flow path 210. The flushing fluid delivery unit 220 comprises a flushing fluid reservoir 221 which receives a biocompatible flushing fluid FF and a flushing fluid delivery unit configured to provide the biocompatible flushing fluid FF to the sample flow path 210. In this embodiment, the flushing fluid delivery unit is an actuator (such as a pump) integrated into the flushing fluid reservoir 221 (and therefore not depicted separately).

The flushing fluid delivery unit 220 is joined to the sample flow path 210 at a first junction between the inlet 210a and the sampling valve 215, such that the flushing fluid delivery unit 220 joins the sample flow path 210 upstream of the sampling valve. A flushing unit valve 225 is provided between the flushing fluid reservoir 221 and the sample flow path 210 which selectively permits fluid flow between the flushing fluid reservoir 221 and the sample flow path 210. The flushing fluid delivery unit 220, and specifically the flushing fluid delivery unit, is therefore configured to provide the biocompatible flushing fluid FF to the sample flow path 210 from the flushing fluid reservoir 221.

The fluidic device 201 further comprises a calibration fluid unit 230 comprising a calibration fluid reservoir 231 for receiving a calibration fluid CF. The calibration fluid reservoir 231 is fluidically connected to the sample flow path 210 at a second junction. In this embodiment, this is at the sampling valve 215. In particular, the sampling valve 215 is a three-way valve which also forms the valve for controlling fluid flow from the calibration fluid reservoir 231 to the sample flow path 210. The calibration fluid unit 230 also comprises a calibration fluid delivery unit configured to provide the calibration fluid CF to the sample flow path 210. In this embodiment, the calibration fluid delivery unit is an actuator (such as a pump) integrated into the calibration fluid reservoir 231 (and therefore not depicted separately).

Downstream of the sensor assembly interface 210b and sensor assembly 260 is a waste reservoir 270 for receiving any waste fluid. Access to this is controlled by a waste valve 275, which selectively allows for fluid flow out of the sample flow path 210 and into the waste reservoir 270.

This fluidic device 201 accordingly primarily differs from that of the embodiment of FIG. 1 in that the first junction is upstream of the sampling valve 215. This device can be operated in the same manner as the fluidic device 101 of FIG. 1, such as set out in FIGS. 3A to 3D, 4A and 4B, although it will be appreciated that the order and configuration of open and closed valves will be different. The fluidic device of FIG. 6 as can enable the upstream part of the sample flow path 210 to be flushed during measurement, as it does not require the sampling valve 215 to be open.

The fluidic devices disclosed herein, such as fluidic device 101 and 201, can be used in sensor systems, such as sensor system 100 to monitor the properties of bodily fluid BF. These may be used to take plural samples of the bodily fluid BF at intervals over a time period during the treatment or process carried out by the associated extracorporeal bodily fluid system, such as the system 199 depicted in FIG. 5. This may be an automated process, for example under the control of the control unit at set and/or regular intervals or may be a manual process. These sampling processes, which will have downtime between measurements (e.g. 1-90 minutes, or more commonly 5-20 minutes) are particularly prone to clogging (such as due to blood clotting) and so the above fluidic device and flushing process is particularly suited to these systems. The flushing may be carried out after each measurement, as can be the steps of providing calibration fluid to the sensor assembly.

For example, one example use of the fluidic device comprises:
connecting the fluidic device to a circulatory fluid flow path of an extracorporeal bodily fluid system, where the sensor assembly is in a dry state (as in storage);
providing calibration fluid to the sensors (transducers) of the sensor assembly (this step may comprise allowing the sensors to hydrate (e.g. for 1-30 mins). Sensor calibration can be performed at the end of this step);
sampling bodily fluid from the circulatory fluid flow path and providing the bodily fluid to the sensor assembly and carrying out the measurements (this may be for 1-90 seconds, for example, such as 30 s); and
flushing bodily fluid from the sensor assembly using flushing fluid for at least an upstream washing step (as set out above) and optionally calibration fluid for the downstream washing step or flushing fluid for a downstream washing step (as set out above). Where flushing fluid is used for the downstream washing step, this step may further comprise providing calibration fluid to the sensors (transducers) of the sensor assembly.

FIG. 7 schematically depicts a method of operating a fluidic device according to an embodiment, the method comprising:
providing a fluidic device 302 comprising:
   a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly;
   a sampling valve provided along the sample flow path, the sampling valve configured to selectively permit fluid to flow along the sample flow path between the inlet and the sensor assembly interface; and
   a flushing unit for flushing bodily fluid from at least a part of the sample flow path, the flushing unit comprising:
      a flushing fluid reservoir for receiving a biocompatible flushing fluid, the flushing fluid reservoir fluidically connected to the sample flow path at a first junction downstream of the inlet; and
      a flushing fluid delivery unit configured to provide the biocompatible flushing fluid to the sample flow path from the flushing fluid reservoir,
   flowing bodily fluid from the inlet to the sensor assembly interface of the sample flow path 304; and
   flushing the sample flow path to remove bodily fluid 306 by flowing biocompatible flushing fluid to flow upstream from the flushing fluid reservoir to the inlet of the sample flow path.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present invention can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

1. A fluidic device for use in an extracorporeal bodily fluid system comprising a circulatory fluid flow path, the fluidic device comprising:
a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly;
a sampling valve provided along the sample flow path, the sampling valve configured to selectively permit fluid to flow along the sample flow path between the inlet and the sensor assembly interface; and
a flushing unit for flushing bodily fluid from at least a part of the sample flow path, the flushing unit comprising:
a flushing fluid reservoir for receiving a biocompatible flushing fluid, the flushing fluid reservoir fluidically connected to the sample flow path at a first junction downstream of the inlet; and
a flushing fluid delivery unit configured to provide the biocompatible flushing fluid to the sample flow path from the flushing fluid reservoir,
wherein the fluidic device is configured such that, in use, the flushing fluid delivery unit causes biocompatible flushing fluid to flow upstream from the flushing fluid reservoir to the inlet of the sample flow path.

2. The fluidic device of claim 1, wherein the first junction is downstream of the sampling valve in the sample flow path.

3. The fluidic device of claim 2, further comprising a sensor assembly control valve provided between the first junction and the sensor assembly interface, the sensor assembly control valve configured to selectively permit fluid to flow along the sample flow path between the first junction and the sensor assembly interface.

4. The fluidic device of claim 1, further comprising a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path.

5. The fluidic device of claim 4, wherein the first junction is upstream of the second junction.

6. The fluidic device of claim 5, wherein a valve is located in the sample flow path between the first and second junctions to selectively permit fluid flow along the sample flow path.

7. The fluidic device of claim 1, wherein the flushing unit is operable to draw fluid from the sample flow path into the flushing fluid reservoir.

8. The fluidic device of claim 1, further comprising a sensor assembly, wherein the sensor assembly interface of the sample flow path is in fluid communication with the sensor assembly.

9. The fluidic device of claim 1, wherein the fluidic device is further configured such that, in use, the flushing fluid delivery unit causes biocompatible flushing fluid to flow downstream from the flushing fluid reservoir through sample flow path.

10. A sensor system for use in an extracorporeal bodily fluid system, comprising:
the fluidic device of claim 1; and
a control unit configured to operate the fluidic device,
wherein the control unit is configured to operate the fluidic device so as to cause biocompatible flushing fluid to flow upstream from the flushing fluid reservoir to the inlet of the sample flow path.

11. The sensor system of claim 10,
wherein the fluidic device further comprises a sensor assembly control valve provided between the first junction and the sensor assembly interface, the sensor assembly control valve configured to selectively permit fluid to flow along the sample flow path between the first junction and the sensor assembly interface; and
wherein the control unit is configured to close the sensor assembly control valve when operating the fluidic device to provide biocompatible flushing fluid to flow upstream from the flushing fluid reservoir to the inlet of the sample flow path.

12. The sensor system of claim 10, wherein the control unit is configured to:
operate the sampling valve to provide bodily fluid from the inlet to the sensor assembly interface; and
subsequently, the control unit is configured to operate the fluidic device so as to cause biocompatible flushing fluid to flow upstream from the flushing fluid reservoir to the inlet of the sample flow path.

13. The sensor system of claim 10,
wherein the flushing unit is operable to draw fluid from the sample flow path into the flushing fluid reservoir; and
wherein the control unit is configured to operate the fluidic device so as to draw fluid from the sample flow path into the flushing fluid reservoir.

14. The sensor system of claim 10,
further comprising a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path; and
wherein the control unit is configured to control delivery of the calibration fluid to the sensor assembly interface.

15. The sensor system of claim 10, wherein the control unit is configured to operate the fluidic device so as to cause biocompatible flushing fluid to flow downstream from the flushing fluid reservoir through sample flow path.

16. A method of operating a fluidic device, the method comprising:
providing a fluidic device comprising:
a sample flow path comprising an inlet and a sensor assembly interface downstream of the inlet, wherein the inlet is for receiving bodily fluid from the circulatory fluid flow path and the sensor assembly interface is for providing bodily fluid to a sensor assembly;
a sampling valve provided along the sample flow path, the sampling valve configured to selectively permit fluid to flow along the sample flow path between the inlet and the sensor assembly interface; and
a flushing unit for flushing bodily fluid from at least a part of the sample flow path, the flushing unit comprising:
a flushing fluid reservoir for receiving a biocompatible flushing fluid, the flushing fluid reservoir fluidically connected to the sample flow path at a first junction downstream of the inlet; and
a flushing fluid delivery unit configured to provide the biocompatible flushing fluid to the sample flow path from the flushing fluid reservoir,
flowing bodily fluid from the inlet to the sensor assembly interface of the sample flow path; and
flushing the sample flow path to remove bodily fluid by flowing biocompatible flushing fluid to flow upstream from the flushing fluid reservoir to the inlet of the sample flow path.

17. The method of claim 16, further comprising:
prior to the flowing bodily fluid from the inlet to the sensor assembly interface of the sample flow path, providing a biocompatible fluid to the sample flow path; and
drawing the biocompatible fluid from the sample flow path into the flushing fluid reservoir,
wherein the biocompatible fluid is used in the subsequent flushing the sample flow path to remove bodily fluid.

18. The method of claim 16,
further comprising providing a sensor assembly fluidly connected to the sensor assembly interface of the sample flow path; and
wherein the flowing bodily fluid from the inlet to the sensor assembly interface of the sample flow path further comprises flowing the bodily fluid over the sensor assembly so that a property of the bodily fluid can be detected.

19. The method of claim 16,
wherein the fluidic device further comprising a calibration fluid unit comprising a calibration fluid reservoir for receiving a calibration fluid, wherein the calibration fluid reservoir is fluidically connected to the sample flow path at a second junction and a calibration fluid delivery unit configured to provide the calibration fluid to the sample flow path; and
wherein the method further comprises providing calibration fluid to the sensor assembly interface.

20. The method of claim 16, wherein flushing the sample flow path to remove bodily fluid further comprises flowing biocompatible flushing fluid to flow downstream from the flushing fluid reservoir through sample flow path.
